(19) United States
(12) Patent Application Publication
(10) Pub. No.: US 2024/0076267 A1
(43) Pub. Date: Mar. 7, 2024

(54) Method for preparing 2-hydroxy-4-methylthiobutyronitrile or the selenium equivalent thereof, and applications

(71) Applicant: ADISSEO FRANCE S.A.S., ANTONY (FR)

(72) Inventors: Virginie BELLIERE-BACA, MILLERY (FR); Aymeric GUINAUDEAU, MILLERY (FR)

(21) Appl. No.: 18/280,388

(22) PCT Filed: Mar. 4, 2022
(86) PCT No.: PCT/FR2022/050388

§ 371 Date: Sep. 5, 2023

(30) Foreign Application Priority Data
Mar. 4, 2021 (FR) FR2102123

(51) Int. Cl. C07C 319/20; C07C 319/28

(52) U.S. Cl. CPC C07C 319/20; C07C 319/28

(57) ABSTRACT

A method for the preparation of the 2-hydroxy-4-methylthiobutyronitrile (HMTBN) or its selenium equivalent (HMSeBN) from hydrocyanic acid and 3-methylthiopropanal (MTP) or the corresponding selenium aldehyde (MSeP) includes the following steps: the molar ratio of the HCN to MTP or to MSeP is adjusted to a value greater than or equal to 1 and the pH is adjusted and maintained at a value greater than or equal to 3.5, to obtain a reaction medium in which the HMTBN or the HMSeBN is formed, then the pH of the reaction medium is lowered to a value less than or equal to 2.5 and the HCN is extracted from the reaction medium, and the HMTBN or the HMSeBN is recovered. The applications of this method are also related.

### TECHNICAL FIELD

The disclosure relates to an improvement in a method for producing 2-hydroxy-4-methylthiobutyronitrile (HMTBN) or 2-hydroxy-4-methylselenobutyronitrile (HMSeBN), from 3-methylthiopropanal (MTP) or 3-methylselenopropanal (MSeP), respectively, and hydrocyanic acid (HCN).

### BACKGROUND

The HMTBN and HMSeBN are precursors for the synthesis of methionine and its selenium equivalent, the selenomethionine. By way of illustration, a synthesis of methionine from HMTBN is described in document WO01/60790A1. By reaction with ammonia, HMTBN is transformed into 2-amino-4-methylthiobutyronitrile (AMTBN) which in turn is reacted with acetone in a basic medium to form 2-amino-4-methylthiobutyramide (AMTBM). A catalytic hydrolysis of AMTBM leads to ammonium methionine from which methionine is recovered.

The HMTBN is also an intermediate in the production of the hydroxy analogue of the methionine, the 2-hydroxy-4-methylthiobutyric acid (HMTBA). For example, according to document US2001/0001105A1, a continuous method for the synthesis of the 2-hydroxy-4-methylthiobutyric acid (HMTBA) from the 2-hydroxy-4-methylthiobutyronitrile (HMTBN) is known, which consists, in a first step, in hydrating the HMTBN to 2-hydroxy-4-methylthiobutyramide (HMTBM) in the presence of an aqueous solution of a mineral acid such as sulfuric acid, then, in a second step, in hydrolyzing the HMTBM to HMTBA. Comparable syntheses can be carried out in the selenium series to lead to the 2-hydroxy-4-methylselenobutyric acid (HMSeBA).

The size of the methionine market no longer needs to be presented, in particular in animal nutrition, and its producing methods are still the subject of numerous developments.

The 2-Hydroxy-4-methylthiobutyric acid (HMTBA), the liquid equivalent of methionine, its salts, its chelates, in particular the metal chelates (of Ca, Zn, Co, Mn, Cu, Fe, Mg . . . ) and its esters, such as the isopropyl and tert-butyl esters of the HMTBA, are also widely used in animal nutrition. Likewise, the selenium derivatives of this acid, of these salts, of these chelates and of these esters are of major interest in animal nutrition.

The preparation of HMTBN from hydrocyanic acid (HCN) and 3-methylthiopropanal (MTP) is well known and widely used on an industrial scale, for example the method disclosed in document US2012/215022A1. This method includes the following steps:
the MTP is reacted with the HCN in the presence of a catalyst selected from amines, in a multizone reactor, to produce a reaction mixture containing the HMTBN formed as well as the reagents, MTP and HCN and the catalyst, and
the unreacted HCN is extracted from the reaction medium to an absorption zone containing MTP and the catalyst, where it reacts again.

According to this method, yields of HMTBN greater than 99 mol % are obtained.

The reaction of MTP with HCN to HMTBN being balanced, the reaction medium always contains, in addition to HMTBN, residual HCN and MTP.

By using an HCN/MTP ratio close to 1 and by extracting the residual HCN from the reaction medium, the method according to US2012/215022A1 makes it possible to improve the profitability of the method. Furthermore, the use of an amine-type catalyst makes it possible to limit the formation of by-products from MTP compared to other basic catalysts.

The disadvantages of these methods are significant residual concentrations of HCN and MTP in the obtained HMTBN.

The presence of residual HCN in the HMTBN leads to the formation of formic acid in the downstream steps responsible for a drop in the quality of the final product and/or an increase in the chemical oxygen demand (COD) of the aqueous effluents making them expensive to treat.

According to EP0601195A1, in the context of the production of 2-hydroxy-4-methylthiobutyric acid (HMTBA) from HMTBN, a step of preparing HMTBN from MTP and HCN is illustrated. The reaction is carried out in an HCN:MTP molar ratio of 1.1, in the presence of NaOH, then sulfuric acid is added to lower the pH to 3. The unreacted HCN is extracted by distillation under pressure, making it possible to limit the formation of the aforementioned formic acid. Nonetheless, the MTP content remains high and affects the purity of the products formed downstream, in particular the HMTB.

The presence of MTP in the reaction medium is indeed just as detrimental because this aldehyde leads to the formation of degradation products of the aldol type, crotomers and derivatives, resulting from reactions of oligomerization of MTP, which contribute just as much as HCN to the decrease in methionine or HMTBA yields and possibly a lower quality of the final product if these products are not extracted.

### SUMMARY

The present disclosure provides a solution making it possible to lower the contents of MTP and HCN in the reaction medium and therefore to reduce the disadvantages associated with their presence, having the consequence of increasing the synthesis yields, avoiding costly treatments of the method effluents and improving the quality of the final product.

The disclosure provides a method for preparing the 2-hydroxy-4-methylthiobutyronitrile (HMTBN) or 2-hydroxy-4-methylselenobutyronitrile (HMSeBN) from 3-methylthiopropanal (MTP) or 3-methylselenopropanal (MSeP), respectively, and hydrocyanic acid (HCN), which comprises at least the following steps:
the molar ratio of HCN to MTP or to MSeP is adjusted to a value greater than or equal to 1 and the pH is adjusted and maintained at a value greater than or equal to 3.5, to obtain a reaction medium in which the HMTBN or HMSeBN is formed,
then the pH of the reaction medium is lowered to a value less than or equal to 2.5 and the HCN is extracted from the reaction medium,
and HMTBN or HMSeBN is recovered.

It has been found that by combining determined values of the ratio of HCN to MTP and determined values of the pH of the reaction medium in the synthesis and extraction of the HCN, respectively, according to the above method, it is managed to promote the consumption of MTP in the reaction medium, which makes it possible to recover a stream of HMTBN or HMSeBN containing no or very little aldehyde and HCN. This method further has a major advantage in that to be implemented, the conventional production line can be adapted without substantial transformation.

The adjustment of the values respectively of the ratio of HCN to MTP and of the pH of the reaction medium, in the first step of the method, is necessary to promote the formation of the HMTBN or the HMSeBN, with a residual MTP concentration as low as possible.

With regard to the ratio of HCN to MTP, it is known that the equilibrium of the reaction leads to a low residual concentration of MTP when it is greater than 1. According to the disclosure, it is determined at a value of at least 1; advantageously this value remains close to 1, it can in particular vary from 1 to 2, the value of 2 having to be considered as maximum since beyond that the excess of HCN is highly detrimental economically. The pH of the reaction medium is determined at a value of at least 3.5, but it is preferably at least 4 and even better still at least 5, for an optimal reaction, which is an important parameter during the implementation of this synthesis on an industrial scale.

Before discussing the disclosure in detail, certain terms and expressions used in this text are defined.

By «value greater than or equal to» or «value of at least», is meant that the upper limit is obviously not unlimited. As previously said, the reaction of the MTP with the HCN is known to those skilled in the art and it is up to their general knowledge to estimate the value beyond which the concerned step can no longer be carried out. Thus, at the first step, the HCN/aldehyde ratio will generally not exceed 2; similarly, the pH of the reaction medium will not exceed 8.

Also, by «value less than or equal to» or «value of at most», those skilled in the art understand that below a lower limit, the concerned step can no longer be carried out. Thus, when the pH value of the reaction medium is lowered to a value of 3.5 or below, it is understood that it cannot go below 0.

By aldehyde in the present text, is meant either the MTP (which is also equivalent to MMP for methyl mercapto propionaldehyde and to AMTP for methylthiopropionic aldehyde) and the MSeP.

### DETAILED DESCRIPTION OF THE DISCLOSURE

Characteristics of the method of the disclosure are mentioned below, they can be considered alone or in combination.

According to an embodiment of the disclosure, the molar ratio of the HCN to MTP is adjusted to a value greater than or equal to 1.02. Below, it is observed that the performance of the method tends to decrease. But advantageously, this value does not exceed 1.5, the required energy to extract the HCN becoming too high compared to the expected gains.

Another advantage of a method of the disclosure lies in the very wide range of temperatures in which it can be carried out. A preferred range is 50° C. to 110° C. Depending on the temperature, HCN is in liquid or gaseous state. In an embodiment, the HCN is supplied in gaseous form into the reaction medium and the temperature of said medium is maintained above 30° C., better still above 50° C., or even above 60° C.

The pressure conditions in the reaction medium are in the range of 1 to 1.5 bara (bar absolute).

The adjustment of the pH to a value of at least 3.5 and its maintaining at this value are generally ensured by a buffer solution. This can be selected from all the suitable pairs to which those skilled in the art have recourse, such as citric acid/sodium citrate, citric acid/caustic soda, sodium citrate/phosphoric acid.

According to a main characteristic of a method of the disclosure, in the second step, the pH of the reaction medium is lowered to a value less than or equal to 2.2, in particular to a value less than or equal to 2, or even to a value less than or equal to 1.5.

The pH of the reaction medium is lowered to a value less than or equal to 2.5 by an acid which the person skilled in the art is able to select on the basis of his skills. It is selected in particular from mineral acids such as sulfuric acid, nitric acid, hydrochloric acid and any mixture thereof.

The HCN can be extracted from the reaction medium by any appropriate technique such as stripping (using a vector gas such as steam, nitrogen, air, CO₂ and any mixture of thereof), evaporation, distillation, membrane methods. In an embodiment of the disclosure, the evaporation is used.

The extraction of the HCN makes it possible to recycle it at the step of reaction with the aldehyde. It can be directly recycled, it can also be treated by one or more operations before being reintroduced into the reaction medium.

The method of the disclosure can be carried out continuously, which is moreover a preferred mode of use of this method.

As previously stated, the applications of a method of the disclosure comprise the production of methionine, selenomethionine, 2-hydroxy-4-methylthiobutyric acid (HMTBA) and 2-hydroxy-4-methylselenobutyric (HMSeBA),

Thus, the disclosure provides a method for the production of methionine or selenomethionine, starting respectively from 2-hydroxy-4-methylthiobutyronitrile (HMTBN) or from 2-hydroxy-4-methylselenobutyronitrile (HMSeBN), the method comprising at least the following steps:
the HMTBN or the HMSeBN is prepared by a method as defined above, generally or in any one of its variants, and
the HMTBN or HMSeBN is converted into methionine or selenomethionine, respectively, by one of the following ways:
a) converting HMTBN or HMSeBN directly into methionine or selenomethionine; by way of example, this conversion can be carried out in the presence of at least water and of a catalyst comprising at least one of alumina, titanium dioxide and zirconia, and optionally, even preferably in presence of ammonia, or
b) reacting HMTBN with NH₃ and CO₂ to produce methionine hydantoin which is saponified with a base such as NaOH, K₂CO₃, to produce Na or K methionine; this methionine is then acidified to form methionine, as described for example in U.S. Pat. No. 5,990,349A, or
c) converting HMTBN or HMSeBN to 2-amino-4-methylthiobutyronitrile (AMTBN) or 2-amino-4-methylselenobutyronitrile (AMSeBN),
the AMTBN or AMSeBN is converted into 2-amino-4-methylthiobutyramide (AMTBM) or 2-amino-4-methylselenobutyramide (AMSeBM), then
the AMTBM or AMSeBM is hydrolyzed to methionine or selenomethionine,
as described for example in WO01/60790A1, or
d) converting HMTBN or HMSeBN into 2-amino-4-methylthiobutyronitrile (AMTBN) or 2-amino-4-methylselenobutyronitrile (AMSeBN),
the AMTBN or AMSeBN is converted into methionine or selenomethionine;

By way of example, the conversion of AMTBN or AMSeBN into methionine or selenomethionine, respectively, according to way d) can be carried out in the presence of at least water and of a catalyst comprising at least one alumina, titanium dioxide and zirconia, and optionally in the presence of ammonia.

The disclosure also provides a method for producing the 2-hydroxy-4-methylthiobutyric acid (HMTBA) or the 2-hydroxy-4-methylselenobutyric acid (HMSeBA), respectively from the 2-hydroxy-4-methylthiobutyronitrile (HMTBN) or the 2-hydroxy-4-methylselenobutyronitrile (HMSeBN), the method comprising at least the following steps:
preparing the HMTBN or the HMSeBN by a method as defined above, generally or in any one of its variants, and
converting the HMTBN or HMSeBN to HMTBA or HMSeBA.

By way of example, the HMTBN or HMSeBN is converted into HMTBA or HMSeBA, in the presence of at least water, a weak acid such as acetic acid, formic acid and propionic acid, and a catalyst comprising at least one of alumina, titanium dioxide and zirconia.

The disclosure and its advantages are illustrated in the examples below.

### Example 1: Synthesis of HMTBN According to the Disclosure in a Molar Ratio of HCN:MTP of 1.1:1

The HMTBN synthesis is carried out by bringing a stream of gaseous HCN containing 10% HCN, 61% N₂, 1% CO₂, 4% CO and 24% water, expressed as mass percentages, into contact with Liquid MTP. The molar ratio is 1.1:1. The medium is buffered at pH=5 with a citric acid/sodium citrate mixture and the reaction takes place at 70° C.

The obtained HMTBN solution contains 70.5% (mass) of HMTBN, 4000 ppm of HCN, 1000 ppm of MTP and 29% (mass) of water.

The pH of this mixture is lowered to 2 by the sulfuric acid. The HCN is extracted from the mixture by evaporation, by heating said mixture to 65° C. at 250 mbar. The final medium contains only 65 ppm of HCN and 1200 ppm of MTP, 72% (mass) of HMTBN and 27.9% (mass) of water.

The vapors are partly condensed. The remaining gases composed of 75% (mass) HCN, 9% (mass) air and 16% (mass) water are returned with the HCN stream to the synthesis.

The liquid composed of 97% (mass) water, 0.2% (mass) MTP and 2.8% (mass) HCN is returned with the MTP to the synthesis.

### Example 2: Synthesis of HMTBN According to the Disclosure in a Molar Ratio of HCN:MTP of 1.05:1

The HMTBN synthesis is carried out by bringing a stream of HCN gas containing 10% HCN, 61% N₂, 1% CO₂, 4% CO and 24% water, expressed as mass percentages, into contact with liquid MTP. The molar ratio is 1.05:1. The medium is buffered at pH=5 with a citric acid/sodium citrate mixture and the reaction takes place at 70° C.

The obtained HMTBN solution contains 70.5% (mass) of HMTBN, 2000 ppm of HCN, 1900 ppm of MTP and 29.1% (mass) of water.

The pH of this mixture is lowered to 2 by sulfuric acid. The HCN is extracted from the mixture by evaporation, by heating the mixture to 65° C. at 250 mbar. The final medium contains only 50 ppm of HCN, 2100 ppm of MTP, 72% (mass) of HMTBN and 27.8% (mass) of water.

The vapors are partly condensed. The remaining compressed gases composed of 66% HCN by weight, 18% air and 16% water are returned with the HCN stream to the synthesis.

The liquid composed of 97% (mass) water, 0.5% (mass) MTP and 2.5% (mass) HCN is returned with the MTP to the synthesis.

### Example 3: Synthesis of HMTBN at Different pH

The synthesis of HMTBN is carried out under the conditions of Example 1 above, with the difference that, once the HMTBN has been obtained, the pH is lowered respectively to 3.4 (for comparison), to 2.2, 2 and 1.5 (according to the disclosure). The pH is lowered by adding sulfuric acid. For each test, the content, before (A) and after removal (B) of HCN from the reaction medium, of HMTBN, HCN, MTP and HMTBM resulting from the hydration of the formed HMTBN, is measured. Those of HMTBN, MTP and HMTBM are determined by HPLC, that of HCN by Raman analysis. The HCN is removed by stripping with nitrogen at a flow rate of 0.5 L/min and collected in a trap containing sodium hydroxide whose HCN content corresponding substantially to the extracted HCN is also determined (C).

The table below shows the obtained results.

TABLE

| Reaction | | HMTBN | HCN | MTP |
|---|---|---|---|---|
| mixture | pH | (% m/m) | (ppm) | (ppm) |
| A | 3.4 | 68 | 4000 | 740 |
| B | | 76 | >200 | 9500 |
| C | | — | 6500 | — |
| A | 2.2 | 70 | 4000 | 800 |
| B | | 81 | nd | 1800 |
| C | | — | 4000 | — |
| A | 2.0 | 70 | 4000 | 800 |
| B | | 76-79 | nd | 1020 |
| C | | — | 3600 | — |
| A | 1.5 | 68 | 4000 | 890 |
| B | | 72 | nd | 1120 |
| C | | — | 4200 | — |

nd means not determined

These results demonstrate that when the pH of the reaction medium is lowered to a pH of less than or equal to 2.5, in particular to a pH of 2.2, 2, or 1.5, the increase in the content of MTP is strongly reduced, thus limiting the formation of MTP degradation products and leading to high quality HMTBN.

It is further observed that at the pH values to which the reaction medium is lowered according to the disclosure, the amount of HCN collected (C) corresponds substantially to the amount of HCN extracted, whereas when the pH is lowered only at 3.4, it is much higher, which means that HCN is reforming at the expense of HMTBN. This observation is also consistent with the measured quantity of MTP.

1. A method for preparing the 2-hydroxy-4-methylthiobutyronitrile (HMTBN) or the 2-hydroxy-4-methylselenobutyronitrile (HMSeBN) from 3-methylthiopropanal (MTP) or 3-methylselenopropanal (MSeP), respectively, and from hydrocyanic acid (HCN), the method including the following steps:
adjusting the molar ratio of HCN to MTP or to MSeP to a value greater than or equal to 1 and adjusting the pH and maintaining at a value greater than or equal to 3.5, to obtain a reaction medium in which the HMTBN or HMSeBN is formed,
then lowering the pH of the reaction medium to a value less than or equal to 2.5 and extracting the HCN from the reaction medium,
and recovering the HMTBN or the HMSeBN.

2. The method according to claim 1, wherein the molar ratio of the HCN to MTP is adjusted to a value greater than or equal to 1.02.

3. The method according to claim 1, wherein the pH is adjusted and maintained at a value greater than or equal to 4.

4. The method according to claim 1, wherein the method is carried out at a temperature ranging from 50 to 110° C.

5. The method according to claim 1, wherein the pH is adjusted and maintained by a buffer solution selected from the group consisting of pairs citric acid/sodium citrate, citric acid/caustic soda, sodium citrate/phosphoric acid.

6. The method according to claim 1, wherein, in the second step, the pH of the reaction medium is lowered to a value less than or equal to 2.2.

7. The method according to claim 1, wherein the pH of the reaction medium is lowered by an acid selected from the group consisting of sulfuric acid, nitric acid, hydrochloric acid and any mixture thereof.

8. The method according to claim 1, wherein the HCN is extracted from the reaction medium by evaporation, stripping, distillation or membrane method.

9. The method according to claim 1, wherein the extracted HCN is recycled in said method.

10. The method according to claim 1, wherein the method is continuously carried out.

11. A method for producing the methionine or the selenomethionine, respectively from the 2-hydroxy-4-methylthiobutyronitrile (HMTBN) or the 2-hydroxy-4-methylselenobutyronitrile (HMSeBN), the method including the following steps
preparing the HMTBN or the HMSeBN by the method as defined in claim 1, and
converting the HMTBN or HMSeBN into methionine or selenomethionine, respectively, by one of the following ways:
a) converting the HMTBN or the HMSeBN directly to methionine or selenomethionine, or
b) reacting the HMTBN with NH₃ and CO₂ to produce methionine hydantoin which is saponified with a base such as NaOH, K₂CO₃, to produce Na or K methionine; this methionine is then acidified to form methionine, or
c) converting the HMTBN or the HMSeBN is converted-into 2-amino-4-methylthiobutyronitrile (AMTBN) or 2-amino-4-methylselenobutyronitrile (AMSeBN), the AMTBN or the AMSeBN is converted into 2-amino-4-methylthiobutyramide (AMTBM) or 2-amino-4-methylselenobutyramide (AMSeBM), then
the AMTBN or AMSeBM is hydrolyzed to methionine or selenomethionine, or
d) converting the HMTBN or the HMSeBN into 2-amino-4-methylthiobutyronitrile (AMTBN) or 2-amino-4-methylselenobutyronitrile (AMSeBN),
converting the AMTBN or the AMSeBN into methionine or selenomethionine.

12. The method according to claim 11, wherein the conversion of the HMTBN or the HMSeBN into methionine or selenomethionine, respectively, according to way a), is carried out in the presence of at least water and a catalyst comprising at least one of alumina, titanium dioxide and zirconia, and optionally in the presence of ammonia.

13. The method according to claim 11, wherein the conversion of the AMTBN or the AMSeBN into methionine or selenomethionine, respectively, according to way d), is carried out in the presence of at least water and a catalyst comprising at least one of alumina, titanium dioxide and zirconia, and optionally in the presence of ammonia.

14. A method for producing the 2-hydroxy-4-methylthiobutyric acid (HMTBA) or 2-hydroxy-4-methylselenobutyric acid (HMSeBA), starting respectively from 2-hydroxy-4-methylthiobutyronitrile (HMTBN) or 2-hydroxy-4-methylselenobutyronitrile (HMSeBN), wherein
the HMTBN or the HMSeBN is prepared by the method as defined in claim 1, and
the HMTBN or the HMSeBN is converted into HMTBA or HMSeBA.

15. The method according to claim 14, wherein the HMTBN or the HMSeBN is converted into HMTBA or into HMSeBA, respectively, in the presence of at least water, a weak acid and a catalyst comprising at least one of alumina, titanium dioxide and zirconia.
